(19) United States
(12) Patent Application Publication
(10) Pub. No.: US 2008/0103451 A1
(43) Pub. Date: May 1, 2008

(54) Self-Securing Bandage for Drain Tubes

(75) Inventors: Donald Paul Schaefer JR., Joplin, MO (US); David Schaefer, Dallas, TX (US)

Correspondence Address: CHALKER FLORES, LLP, 2711 LBJ FRWY, Suite 1036, DALLAS, TX, 75234, US

(73) Assignee: SCHAEFER MEDICAL, LLC, Dallas, TX (US)

(21) Appl. No.: 11/929,530

(22) Filed: Oct. 30, 2007

Related U.S. Application Data
(60) Provisional application No. 60/855,440, filed on Oct. 31, 2006.

(51) Int. Cl. A61M 25/02

(52) U.S. Cl. 604/180; 604/174

(57) ABSTRACT

The present invention includes methods, kits and bandages that accommodate a medical device extending from an incision in a patient. The bandage includes an outer surface, inner absorbent layer and one or more closure mechanisms. The outer surface includes a slit extending from an outer peripheral edge to an inner peripheral edge that forms an aperture to accommodate a medical device. The inner absorbent layer is in contact with the outer surface and at least partially spans the aperture to accommodate the medical device. The one or more closure mechanisms are used to substantially seal the slit. The bandage is positioned about the incision and secured about the medical device for absorption.

### CROSS-REFERENCE TO RELATED APPLICATIONS

This application claims priority to U.S. Provisional Application Ser. No. 60/855,440, filed Oct. 31, 2006, the contents of which is incorporated by reference herein in its entirety.

### TECHNICAL FIELD OF THE INVENTION

The present invention relates generally to the field of medical dressings, and, more particularly, to self securing surgical dressings for drain tubes.

### BACKGROUND OF THE INVENTION

Surgery by its very nature is a traumatic procedure from which the body must heal. As this healing process continues the body undergoes bleeding, swelling and fluid accumulation as various cells (e.g., white blood cells and scavenger cells) are attracted to the region to begin the process of cleaning up debris, repairing and regenerating the damaged areas. As a result, it is often necessary to position a medical device leading into a patient.

For example after a thoracic surgery, a chest tube is positioned in the thoracic area of a patient for drainage. The chest tube is anchored to a patient by heavy suturing followed by winding and tying the suture around the chest tube. It is imperative that the chest tube be secured to prevent inadvertent removal, which is not only painful to the patient but also dangerous. The thoracic cavity is under negative pressure with respect to the atmosphere and it is possible for atmospheric air to enter the thoracic cavity and present dangers for the patient.

Generally, a chest tube is inserted through an incision made in the patient at the time of treatment (either in surgery or in the emergency situation, e.g., battlefield, emergency room, ambulance or the like. The skin incision from which the chest tube egresses from the chest cavity is carefully sealed to prevent infection and atmospheric exposure of the chest cavity. A bandage is placed over the incision to capture fluids that are excreted about the chest tube and the incision. The bandage must be secured to the chest tube and incision. The bandage replacement process includes positioning the bandage about the chest tube and wrapping, looping and/or tying the bandage around the chest tube. In fact, many doctors develop their own style or method of wrapping, looping and/or tying the bandage. This process is repeated each time the bandage is replaced and becomes very time consuming. After drainage has stopped, the chest tube may be removed from the chest and the opening sealed so that no air will enter the chest. The removal process includes the removal of all gauze, tape and sutures used during the surgery and insertion process. The chest tube is then quickly removed from the chest within a few seconds of the removal of the sutures. The incision is covered by an occlusive dressing that generally includes a sterile gauze with several wide strips of adhesive tape over the sterile gauze to cover the incision.

Given the importance of the chest tube and the possibility of the inadvertent removal of the chest tube, partial removal of the chest tube, dislodging of the chest tube or other complications during bandage changing many doctors do not allow nurses or assistants to apply or change drainage tube (especially chest tube) bandages. Instead, a trained physician must remove the drainage tube bandage, position the new bandage and secure it to the drainage tube by wrapping, looping and/or tying. Each time the bandage is replaced this process must be repeated and inspected to insure the drainage tube is secure, the incision is covered and the bandage itself is secure.

While there are devices and bandages that anchor to drainage tubes and other medical devices, they all have drawbacks and are not anchored in a manner applicable to tubular devices (including drainage tubes) and/or require special dexterity or skill (e.g., skilled doctor) to apply. For example, U.S. Pat. No. 4,221,215 entitled “Anchoring and occluding surgical dressing” discloses a surgical dressing used to anchor medical devices. It is used to secure a chest tubes to a patient and occlude any cut made in a patient for accommodating such medical device. The anchoring device includes elongate bands connected to the dressing and to the medical device to connect and secure the medical device to the dressing. The dressing includes a dressing upper portion and a dressing lower portion. The dressing has adhesive on a rear surface of the dressing upper portion for attachment of the dressing to a patient. Once the medical device is removed from the patient the adhesive on the front surface of the dressing lower portion allows the dressing lower portion to be folded over, superimposed and secured with the upper portion of the dressing to occlude the cut made in the patient for accommodating the medical device.

Similarly, U.S. Pat. No. 3,918,446 entitled “Securement device for intravenous catheter and its tubing” discloses a device for securing to the skin and protecting an infusion needle or catheter in the body portion of a patient and for holding a portion of the excess tubing while providing a cushion between the catheter-tubing assembly and the skin, consisting of a bottom pad and an associated upper pad, both pads being formed of flexible material. However, the securement device is directed to holding small intravenous type devices in place, not for use with drainage tubes and is not amenable to provide the necessary adsorption or sealing.

U.S. Pat. No. 5,569,207 entitled “Hydrocolloid dressing” discloses a dressing having a downwardly extending lip member that is sized to be received adjacent to a percutaneous device which is at least partially implanted into a patient. Furthermore, the partial implantation of the device may result in additional complications, e.g., the risk of exposure of atmospheric air to the thoracic cavity during replacement. Again, the device is not amenable for use with drainage tubes and cannot provide the necessary adsorption or sealing.

U.S. Pat. No. 5,807,341 entitled “Medical catheter dressing device” discloses a medical apparatus and method for managing one or more conveyance tubes used to inject or remove fluids, solids or semi-solids from a patient's body without the need for sutures. This device and similar single opening devices are not amenable for use with drainage tubes as the drainage tube must be disconnected to replace the bandage.

As a result, of the life threatening importance of chest tubes and other medical devices that are partially within the body of a patient, most physicians do not allowing nurses or other care givers change the associated bandage and personally change the associated bandage every time. This procedure includes the removal of the old bandage, by untying or in some cases cutting, with care and precision while being careful not to dislodge the medical device. A new bandage is then positioned about the device and looped and tied, while again being careful to secure the bandage without dislodging the medical device.

Accordingly, there is a need for a bandage/dressing that will accommodate a drainage tube or other medical device while remaining easy to position and secure about the drainage tube or other medical device without removal or disconnection of the drainage tube or other medical device, allowing a nurse or technician to remove and replace the bandage.

### SUMMARY OF THE INVENTION

The present inventors recognized the need for a bandage or dressing that would accommodate a drainage tube, trocart, catheter or other medical device while remaining easy to position and secure and would not require the removal or disconnection of the medical device for bandage removal or replacement.

For example, the present invention provides a disposable surgical bandage to accommodate a medical device extending from an incision in a patient. The bandage allows removal of the bandage and redressing of the incision without the need for complicated securing methods and devices. The disposable surgical bandage includes one or more absorbent layers each having a slit extending from an outer peripheral edge to an inner peripheral edge. The inner peripheral edge forms an aperture to accommodate a medical device. In addition, the disposable surgical bandage includes one or more closure mechanisms to secure substantially the slit. In operation, the medical device is positioned through the slit to the aperture and the bandage is secured about the medical device for absorption of a wound exudates.

The disposable surgical bandage may also include one or more outer surfaces disposed substantially coplanar to the one or more absorbent layers, an outer slit and an outer aperture that aligns with the slit and aperture to allow the medical device to be positioned through the slit to the aperture. There may be a variety of closure mechanism (e.g., loop and hook fastener, an adhesive, a flap, a tape, a second surgical bandage or a combination thereof) and may include different mechanisms on the same disposable surgical bandage. For example, the attachment mechanism may be disposed on the outer surface of the bandage. Another example of a closure mechanism includes a second absorbent layer having an opening extending orthogonally to the slit and extending from an outer peripheral edge to an inner peripheral edge, wherein orthogonal orientation of the opening to the slit secures the medical device and absorbs wound exudates.

The present invention provides a method of bandaging a medical device extending from an incision in a patient by moving a disposable surgical bandage to contact the medical device extending from an incision in the patient. The disposable surgical bandage includes an outer surface having a slit extending from an outer peripheral edge to an inner peripheral edge. The inner peripheral edge forms an aperture to accommodate the medical device. One or more absorbent layers are in contact with the outer surface and at least partially span the aperture to accommodate the medical device. The disposable surgical bandage includes one or more closure mechanisms to cover substantially the slit. The slit is then substantially sealed by the one or more closure mechanisms. In addition, the method includes adhering one or more flaps about the aperture to the medical device to secure the surgical bandage to the medical device for absorption.

A kit for a disposable surgical bandage to accommodate a medical device extending from an incision in a patient is also provided by the present invention. The kit includes a disposable surgical bandage having an outer surface with a slit extending from an outer peripheral edge to an inner peripheral edge. The inner peripheral edge forms an aperture to accommodate a medical device. The surgical bandage includes one or more absorbent layers in contact with the outer surface and at least partially spans the aperture to accommodate the medical device and one or more closure mechanisms to close substantially the slit. The bandage is positioned and secured about the medical device and incision for absorption of wound exudates. In addition, at least one set of step-by-step instructions are included. Furthermore, the kit further includes one or more selected from antibacterial soap, one or more pair of gloves, one or more gauze pads, a post-surgical drain tube, a tape within a roll and one or more waste bags.

### BRIEF DESCRIPTION OF THE DRAWINGS

For a more complete understanding of the features and advantages of the present invention, reference is now made to the detailed description of the invention along with the accompanying figures and in which:

FIG. 1 is a top view of a disposable surgical bandage in accordance with one embodiment of the present invention;

FIG. 2 is a perspective view of a cross section of the disposable surgical bandage in accordance with one embodiment of the present invention;

FIG. 3 is a cross sectional view of the disposable surgical bandage in accordance with one embodiment of the present invention;

FIG. 4 is a top view of a disposable surgical bandage in accordance with one embodiment of the present invention;

FIGS. 5a and 5b are top views of a disposable surgical bandage in accordance with one embodiment of the present invention;

FIG. 6 is a view of a disposable surgical bandage in accordance with one embodiment of the present invention;

FIG. 7 is a perspective view of a disposable surgical bandage in accordance with one embodiment of the present invention;

FIG. 8a is a perspective view and 8b is a top view of a disposable surgical bandage in accordance with one embodiment of the present invention;

FIG. 9a is a top view of a disposable surgical bandage in accordance with one embodiment of the present invention;

FIG. 9b is a top view of a disposable surgical bandage with a medical device extending from an incision in a patient; and

FIGS. 10a-10k are images that illustrate different embodiments of the closure mechanisms of the disposable surgical bandage.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “Bioactive agent” refers to a substance which may be used in connection with an application that is therapeutic or diagnostic. “Bioactive agent” refers to substances which are capable of exerting a biological effect in vitro and/or in vivo.

The term “absorbent article” refers to articles which absorb and contain body exudates, such as body fluid. More specifically, the term refers to articles which are placed against or in proximity to the body of the wearer to absorb and contain the various exudates discharged from the body. The term “disposable” refers to articles which are intended to be discarded after a single use.

The present invention provides a disposable surgical bandage to accommodate a medical device extending from an incision in a patient. The surgical bandage includes an outer surface, one or more absorbent layers and one or more closure mechanisms to accommodate a medical device positioned through the slit to the aperture and the bandage secured about the medical device for absorption. The outer surface includes a slit extending from an outer peripheral edge to an inner peripheral edge. The inner peripheral edge forms an aperture to accommodate a medical device. The one or more absorbent layers are disposed substantially coplanar with the outer surface and at least partially span the aperture to accommodate the medical device. The one or more closure mechanisms secure substantially the slit and in turn the medical device.

Generally, the bandage has an absorbent layer substantially enclosed by an outer layer for placement over an incision. The bandage has an opening that extends to the interior to accommodate a medical device extending from the incision. The opening can be closed by a securing mechanism to secure the bandage about the medical device.

One embodiment includes a bandage having three layers, a central absorbent layer surrounded on each side by a thin outer layer. The bandage has an opening extending through the three layers and into the interior portion of the bandage. A sealable flap is positioned over the opening to close it.

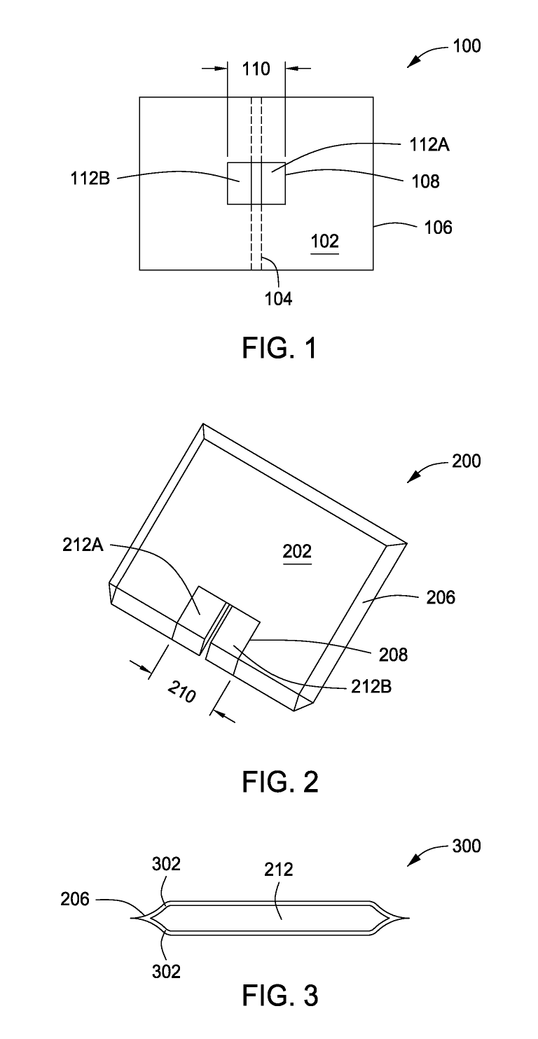

Now referring to FIG. 1, a top view of a disposable surgical bandage 100 to accommodate a medical device (not shown) extending from an incision in a patient (not shown) in accordance with one embodiment of the present invention is shown. The disposable surgical bandage 100 includes an outer surface 102 having a slit 104 extending from an outer peripheral edge 106 to an inner peripheral edge 108 that forms an aperture 110 to accommodate a medical device (not shown). The aperture 110 may be designed of sufficient size, spatial arrangement and orientation to accommodate a variety of medical devices. An inner absorbent layer 112a and 112b is in contact with the outer surface 102 and at least partially spans the aperture 110 to accommodate the medical device (not shown). The disposable surgical bandage 100 also includes one or more closure mechanisms (not shown) to substantially seal the slit 104 and connect the adjacent portions of the outer surface 102. The outer surface 102 may be constructed from a variety of materials including a “felted” material, woven, nonwoven and/or spunbond materials including natural fibers (e.g., wood or cotton fibers), synthetic fibers (e.g., polymeric fibers, polyester, polypropylene, or polyethylene fibers), rayon bonded, carded web or a combination thereof. The absorbent material 46 may contain moisture capturing powders or gels. The absorbent material 46 may also contain antiseptic or antibiotic material.

FIG. 2 is a perspective view of a cross section of the disposable surgical bandage 200 to accommodate a medical device extending from an incision in a patient in accordance with one embodiment of the present invention. The disposable surgical bandage 200 includes an outer surface 202 having an outer peripheral edge 206 to an inner peripheral edge 208 that forms an aperture 210 to accommodate a medical device (not shown). An inner absorbent layer 212a and 212b is in contact with the outer surface 202 and at least partially spans the aperture 210 to accommodate the medical device (not shown). The outer surface 202 may be any woven or non-woven material which facilitates the attachment of pressure sensitive adhesives, tapes or the like. Similarly, the inner absorbent layer 212 may be any woven or non-woven material which facilitates absorption, e.g., gauze. For example, suitable woven and non-woven materials include natural fibers (e.g., wood or cotton fibers), synthetic fibers (e.g., polymeric fibers, polyester, polypropylene, or polyethylene fibers) or a combination of natural and synthetic fibers.

FIG. 3 is a cross sectional view of the disposable surgical bandage 300 to accommodate a medical device extending from an incision in a patient in accordance with one embodiment of the present invention. The disposable surgical bandage 300 includes an outer surface 302 extending on the top and bottom of an inner absorbent layer 312 that is connected at the outer peripheral edge 306. It is to be understood, however, that the mentioned dimensions are only exemplary and can be varied as desired as long as the structure retains its overall function. The disposable surgical bandage 300 may be rectangular (e.g., 5×5, 4×4, 3×3, 2×2, 1×1, 5×1, 4×1, 3×1, 2×1, 5×2, 4×2, 3×2, 5×3, 4×3, 5×4 and incremental variations thereof), oval (e.g., 5×5, 4×4, 3×3, 2×2, 1×1, 5×1, 4×1, 3×1, 2×1, 5×2, 4×2, 3×2, 5×3, 4×3, 5×4 and incremental variations thereof), polygonal, free-formed or any other shape desired. Generally, the disposable surgical bandage 300 may be any size desired for the particular application and range from 0.5 to 7 inches by 0.5 to 7 inches. The disposable surgical bandage 300 may be of any thickness necessary (e.g., 0.1 to 7 inches) and may be of any combination of layers to accommodate that thickness. The aperture 310 may also be of any size and need not be of any specific shape to accommodate the corresponding medical device (not shown). A portion or the entire disposable surgical bandage 300 can be impregnated with a suitable material, polymeric material or layer of polymeric films laminated with the gauze layers.

Although the various embodiments depicted herein include an outer peripheral edge 306 that is sealed, an unsealed outer peripheral edge may also be used. The outer peripheral edge 306 may be open and the various layers held in position with another mechanism, e.g., needlepunching, adhesive, fusion, weaving and so forth. In addition, the outer surface 302 may be on one side of the inner absorbent layer 312 or multiple sides of the inner absorbent layer 312. The outer surface 302 may be varied from one area to another.

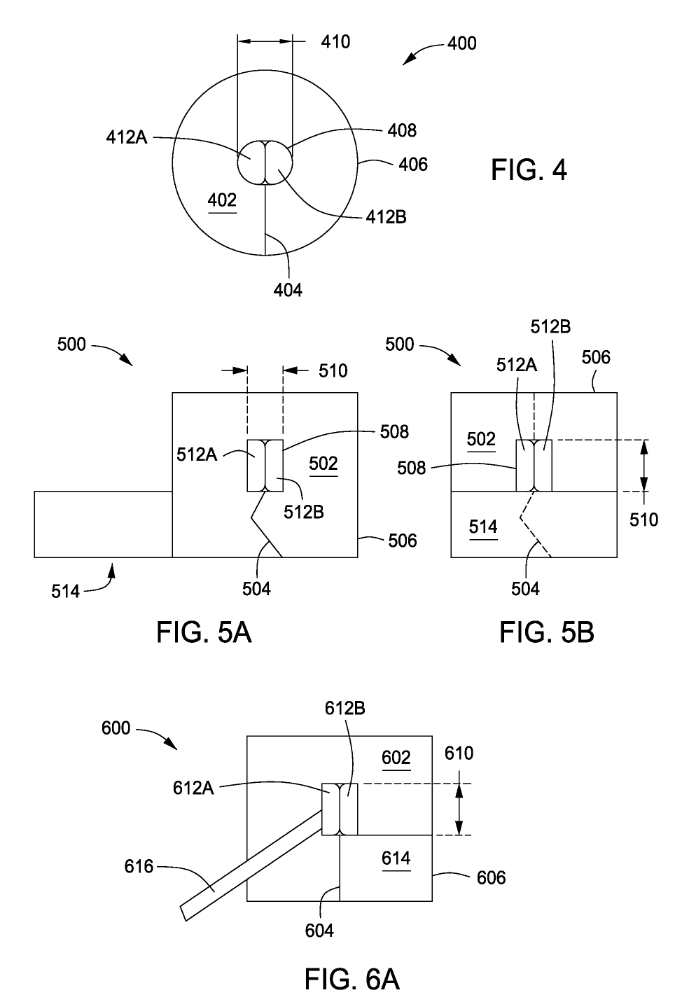

FIG. 4 is a top view of a disposable surgical bandage 400 to accommodate a medical device (not shown) extending from an incision in a patient (not shown) in accordance with one embodiment of the present invention. The disposable surgical bandage 400 includes an outer surface 402 having a slit 404 extending from an outer peripheral edge 406 to an inner peripheral edge 408 that forms an aperture 410 to accommodate a medical device (not shown). An inner absorbent layer 412a and 412b is in contact with the outer surface 402 and at least partially spans the aperture 410 to accommodate the medical device (not shown). The disposable surgical bandage 400 also includes one or more closure mechanisms (not shown) to substantially seal the slit 404 and connect the adjacent portions of the outer surface 402.

FIGS. 5a and 5b are top views of a disposable surgical bandage 500 to accommodate a medical device (not shown) extending from an incision in a patient (not shown) in accordance with one embodiment of the present invention. The disposable surgical bandage 500 includes an outer surface 502 having a slit 504 extending from an outer peripheral edge 506 to an inner peripheral edge 508 that forms an aperture 510 to accommodate a medical device (not shown). An inner absorbent layer 512a and 512b is in contact with the outer surface 502 and at least partially spans the aperture 510 to accommodate the medical device (not shown). The disposable surgical bandage 500 also includes one or more closure mechanisms 514 to substantially seal the slit 504 and connect the adjacent portions of the outer surface 502. The one or more closure mechanisms 514 may be a flap that is folded over the slit 504 and attaches to the outer surface 502. FIG. 5b illustrates the connection of the one or more closure mechanisms 514 to the outer surface 502 on each side of the slit 504 to secure the outer surface 502 about the medical device (not shown). For example, the one or more closure mechanisms 514 may be a flap attached to the outer surface 502 at one point and folded over the slit 504 and attached to the outer surface 502 via tape or adhesive that is generally non-allergenic and non-irritating to humans and animals, e.g., tapes, thin plastics; polymers (e.g., polyvinyl, polypropylene, polyurethane or polyester), fabrics (e.g., cotton, nylon, silk or other naturally occurring or synthetic fabrics), silicon, latex, rubber, acetate products, paper, cellulose, fiber-based material or a combination thereof.

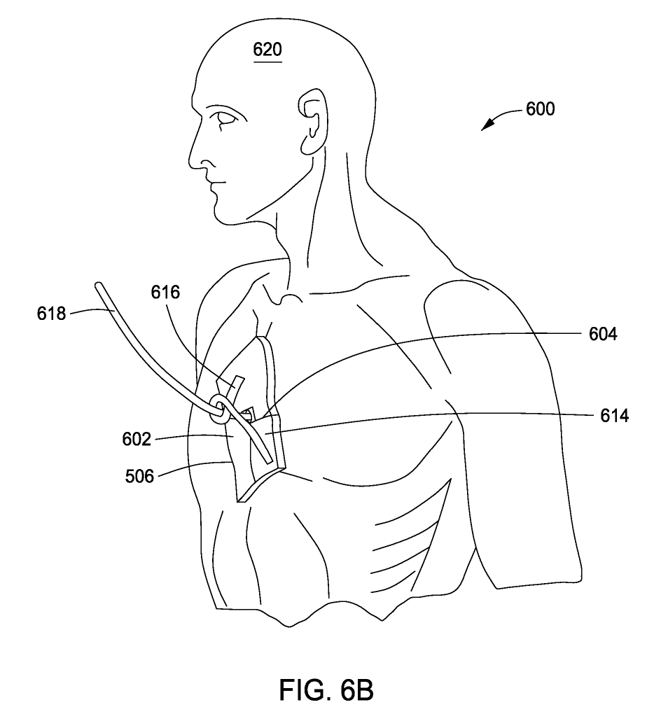

FIG. 6a is a top view of a disposable surgical bandage 600 to accommodate a medical device (not shown) extending from an incision in a patient (not shown) in accordance with one embodiment of the present invention. The disposable surgical bandage 600 includes an outer surface 602 having a slit 604 extending from an outer peripheral edge 606 to an inner peripheral edge 608 that forms an aperture 610 to accommodate a medical device (not shown). An inner absorbent layer 612a and 612b is in contact with the outer surface 602 and at least partially spans the aperture 610 to accommodate the medical device (not shown). The disposable surgical bandage 600 also includes one or more closure mechanisms 614 to substantially seal the slit 604 and connect the adjacent portions of the outer surface 602. The one or more closure mechanisms 614 may be a flap that is folded over the slit 604 and attaches to the outer surface 602. The outer surface 602 also includes a second flap 616 that can be attached to the medical device (not shown) and optionally back to the outer surface 602. FIG. 6b is an image of the disposable surgical bandage 600 positioned to accommodate a medical device 618 extending from an incision in a patient 620 in accordance with one embodiment of the present invention. The medical device may be any device that at least partially extends into/from a portion of the body, e.g., trocart, catheter, drainage tube, discharge tube or the like.

In addition, disposable surgical bandage 600 may include an adhesive on the side facing the patient. In those embodiments, the area around the surgical incision and medical device should be made dry to ensure good adhesion of the adhesive and disposable surgical bandage 600. Immediately prior to placement of the disposable surgical bandage 600 a substance (e.g., tincture of benzoin that may improve the adhesiveness of the disposable surgical bandage 600) may be applied to the surrounding skin.

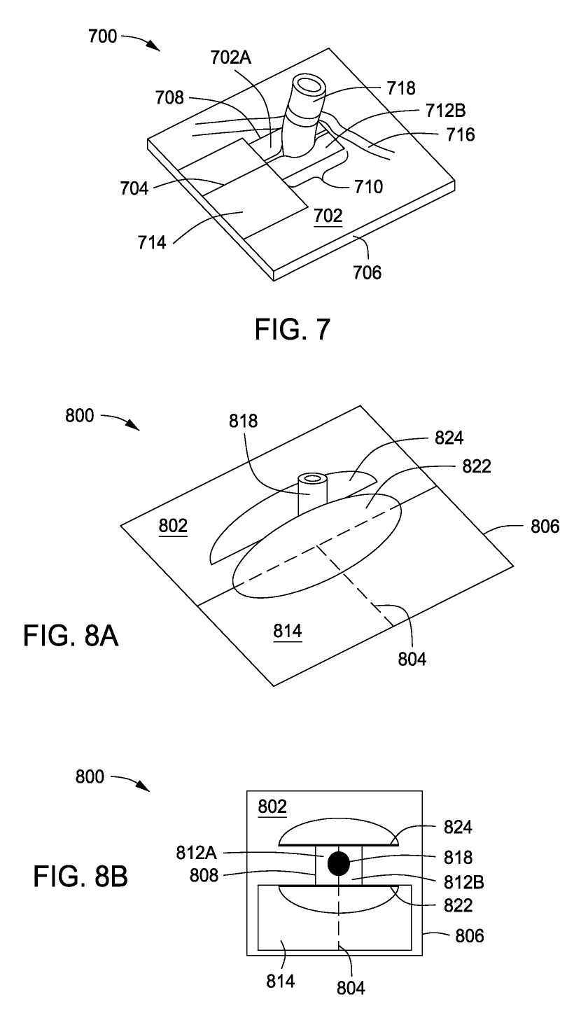

FIG. 7 is a perspective view of a disposable surgical bandage 700 to accommodate a medical device 718 extending from an incision in a patient (not shown) in accordance with one embodiment of the present invention. The disposable surgical bandage 700 includes an outer surface 702 having a slit 704 extending from an outer peripheral edge 706 to an inner peripheral edge 708 that forms an aperture 710 to accommodate a medical device 718. An inner absorbent layer 712a and 712b is in contact with the outer surface 702 and at least partially spans the aperture 710 to accommodate the medical device 718. The disposable surgical bandage 700 also includes one or more closure mechanisms 714 to substantially seal the slit 704 and connect the adjacent portions of the outer surface 702. The one or more closure mechanisms 714 is a flap that is folded over the slit 704 and attaches to the outer surface 702. The outer surface 702 also includes a second flap 716 that can be wrapped and/or affixed to the medical device 718 and attached to the outer surface 702.

FIG. 8a is a perspective view of a disposable surgical bandage 800 to accommodate a medical device 818 extending from an incision in a patient (not shown) in accordance with one embodiment of the present invention is shown. The disposable surgical bandage 800 includes an outer surface 802 having a slit 804 extending from an outer peripheral edge 806 to an inner peripheral edge (not shown) that forms an aperture (not shown) to accommodate a medical device 818. The disposable surgical bandage 800 also includes one or more closure mechanisms 814 to substantially seal the slit 804 and connect the adjacent portions of the outer surface 802. The one or more closure mechanisms 814 may be a flap that is folded over the slit 804 and attaches to the outer surface 802. The outer surface 802 also includes a first aperture flap 822 and second aperture flap 824 attached to the outer surface 802 and can be affixed together around the medical device 818 and/or affixed to the medical device 818.

FIG. 8b is a top view of a disposable surgical bandage 800 to accommodate a medical device 818 extending from an incision in a patient (not shown) in accordance with one embodiment of the present invention. The disposable surgical bandage 800 includes an outer surface 802 having a slit 804 extending from an outer peripheral edge 806 to an inner peripheral edge 808 that forms an aperture 810 to accommodate a medical device 818. An inner absorbent layer 812a and 812b is in contact with the outer surface 802 and at least partially spans the aperture 810 to accommodate the medical device 818. The disposable surgical bandage 800 also includes one or more closure mechanisms 814 to substantially seal the slit 804 and connect the adjacent portions of the outer surface 802. The one or more closure mechanisms 814 is a flap that is folded over the slit 804 and attaches to the outer surface 802. The outer surface 802 also includes a first aperture flap 822 and second aperture flap 824 attached to the outer surface 802 and can be affixed together around the medical device 818 and/or affixed to the medical device 818. The first aperture flap 822 and second aperture flap 824 extend from the outer surface 802 to cover a portion of the medical device 818 extending from the patient (not shown).

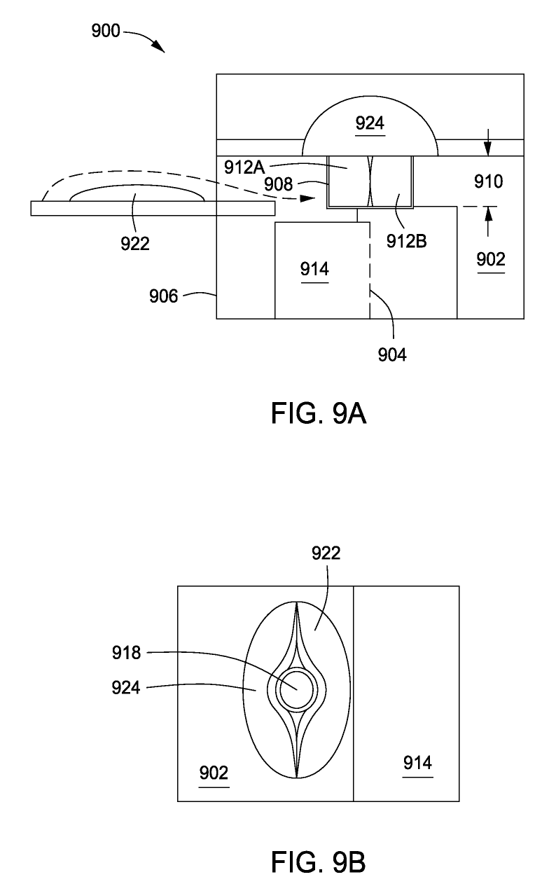

FIG. 9a is a top view of a disposable surgical bandage 900 to accommodate a medical device (not shown) extending from an incision in a patient (not shown) in accordance with one embodiment of the present invention. The disposable surgical bandage 900 includes an outer surface 902 having a slit 904 extending from an outer peripheral edge 906 to an inner peripheral edge 908 that forms an aperture 910 to accommodate a medical device (not shown). An inner absorbent layer 912a and 912b is in contact with the outer surface 902 and at least partially spans the aperture 910 to accommodate the medical device (not shown). The disposable surgical bandage 900 also includes one or more closure mechanisms 914 to substantially seal the slit 904 and connect the adjacent portions of the outer surface 902. The one or more closure mechanisms 914 is a flap that is folded over the slit 904 and attaches to the outer surface 902. The outer surface 902 also includes a first aperture flap 922 and second aperture flap 924 attached to the outer surface 802 and can be affixed together around the medical device (not shown) and/or affixed to the medical device (not shown). The first aperture flap 922 is attached to allow the disposable surgical bandage 900 to be positioned about the medical device (not shown). The one or more closure mechanisms 914 can be folded over the slit 904 and attached to the outer surface 902. The second aperture flap 924 can be positioned on the outer surface 902 to cover a portion of the medical device (not shown). The first aperture flap 922 and the second aperture flap 924 can be adhered about the medical device (not shown).

FIG. 9b is a top view of a disposable surgical bandage 900 to accommodate a medical device 918 extending from an incision in a patient (not shown) in accordance with FIG. 9a. FIG. 9b illustrates the one or more closure mechanisms 914 folded over the slit 904 and attached to the outer surface 902. The first aperture flap 922 is positioned on the outer surface 902 and covers a portion of the medical device. The second aperture flap 924 is also positioned on the outer surface 902 and covers a portion of the medical device. The first aperture flap 922 and the second aperture flap 924 also contact each other to secure the medical device and to close the opening around the medical device.

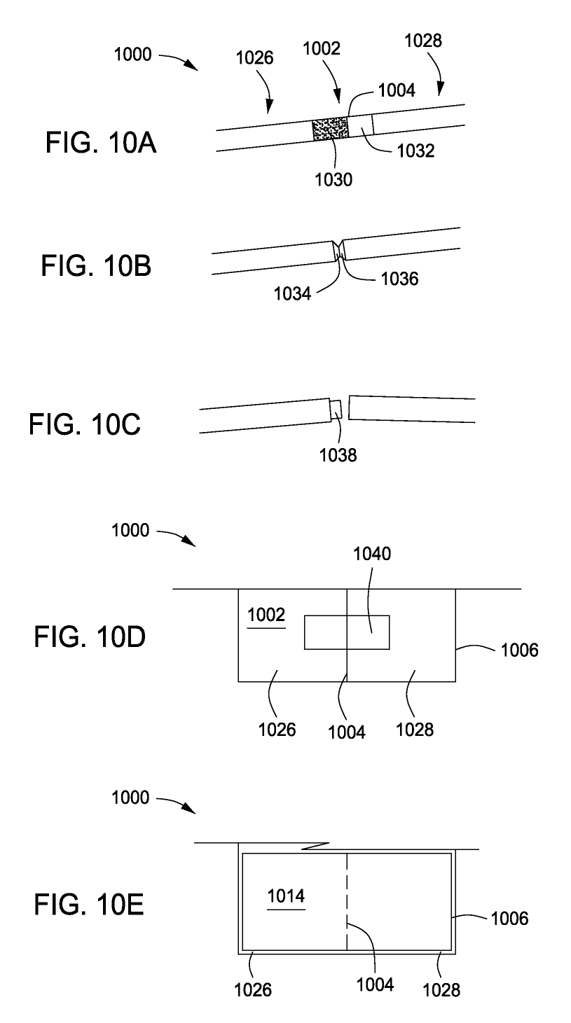

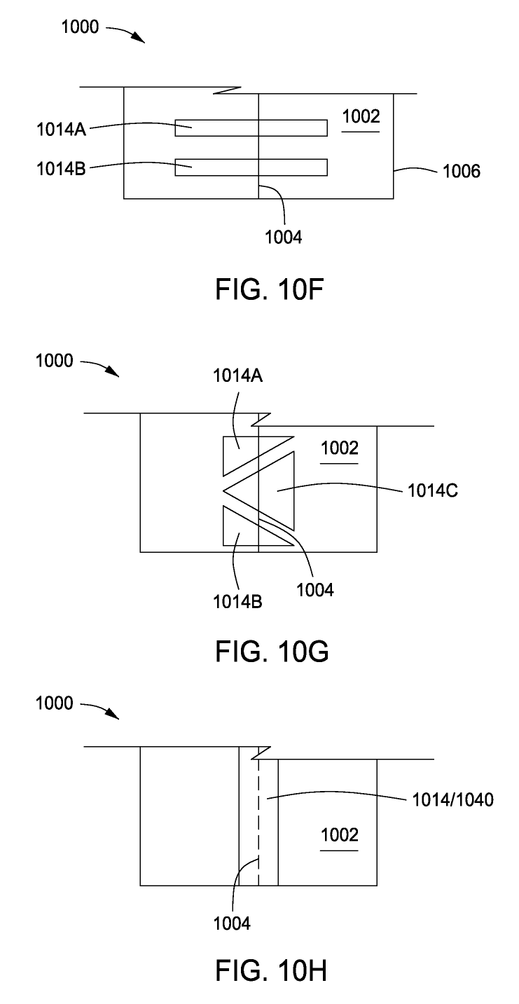

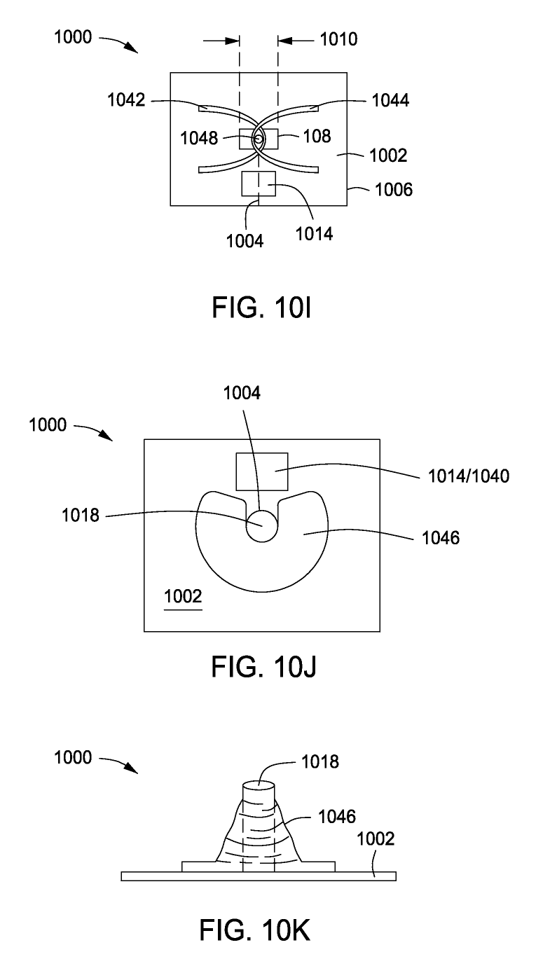

FIGS. 10a-10k are images that illustrate different embodiments of the closure mechanisms of the disposable surgical bandage. FIGS. 10a-10c are side views looking at the slit 1004 that allows the positioning of the medical device. The disposable surgical bandage 1000 includes an outer surface 1002 with a slit 1004 that separates a left portion 1026 and a right portion 1028 of the outer surface 1002.

FIG. 10a illustrates a loop and hook closure mechanism. A loop 1030 is positioned about the right portion 1028 of the slit 1004 that contacts a hook 1032 positioned about the left portion 1026 of the slit 1004. The loop 1030 and hook 1032 connect to close the slit 1004 and secure the surgical bandage 1000 about the medical device (not shown).

FIG. 10b illustrates an adhesive closure mechanism. A first adhesive 1034 is positioned about the right portion 1028 of the slit 1004 that contacts the left portion 1026 of the slit 1004. The first adhesive 1034 connects to close the slit 1004 and secure the surgical bandage 1000 about the medical device (not shown). A protective covering may be placed over the closure mechanism until it is ready for use. Alternatively, a second adhesive 1036 can be placed on the right portion 1028. The first adhesive 1034 and second adhesive 1036 may be the same or different compositions applied directly to the surface, applied to a strip that is in turn applied to the surface, a 2-part epoxy, (e.g., glue, sealant and the like) that is applied individually to each surface.

FIG. 10c illustrates a tape closure mechanism. A tape strip 1038 is positioned about the right portion 1028 of the slit 1004 that contacts the left portion 1026 of the slit 1004. The tape strip 1038 connects to close the slit 1004 and secure the surgical bandage 1000 about the medical device (not shown). Alternatively, a second tape strip (not shown) can be placed on the left portion 1026. A protective covering may be placed over the tape strip 1038 until it is ready for use.

FIGS. 10d-10h are top views of a portion of the surgical bandage looking at the slit 1004 that allows the positioning of the medical device. The disposable surgical bandage 1000 includes an outer surface 1002 having a slit 1004 extending from an outer peripheral edge 1006 to an inner peripheral edge (not shown) that forms an aperture (not shown) to accommodate a medical device (not shown).

FIG. 10d illustrates a tape strip 1040 closure mechanism. A tape strip 1040 is positioned about the right portion 1028 of the slit 1004 and contacts the left portion 1026 of the slit 1004. The tape strip 1040 closes the slit 1004 and secures the surgical bandage 1000 about the medical device (not shown). The tape strip 1040 may be of any dimensions necessary to close the slit 1004.

FIG. 10e illustrates a flap closure mechanism. A flap 1014 is positioned about the right portion 1028 of the slit 1004 and contacts the left portion 1026 of the slit 1004. The flap 1014 closes the slit 1004 and secures the surgical bandage 1000 about the medical device (not shown). The flap 1014 may be of any dimensions necessary to close the slit 1004 and may be constructed from any material applicable to the use.

FIG. 10f illustrates a bi-flap closure mechanism. A first flap 1014a is positioned about the right portion 1028 of the slit 1004 and contacts the left portion 1026 of the slit 1004. A second flap 1014b is positioned about the left portion 1026 of the slit 1004 and contacts the right portion 1028 of the slit 1004. The flaps 1014a and 1014b closes the slit 1004 and secure the surgical bandage 1000 about the medical device (not shown). Alternatively, tape strips may be used in-place of or in addition to the flaps.

FIG. 10g illustrates a multi-flap closure mechanism. A first flap 1014a and second flap 1014b are positioned about the right portion 1028 of the slit 1004 and contacts the left portion 1026 of the slit 1004. A third flap 1014c is positioned about the left portion 1026 of the slit 1004 and contacts the right portion 1028 of the slit 1004. The flaps 1014a, 1014b and 1014c close the slit 1004 and secure the surgical bandage 1000 about the medical device (not shown). Alternatively, tape strips may be used in place of or in addition to the flaps.

FIG. 10h illustrates a tape strip 1040/flap 1014 closure mechanism. A tape strip 1040/flap 1014 is positioned about the right portion 1028 of the slit 1004 and contacts the left portion 1026 of the slit 1004. The tape strip 1040/flap 1014 closes the slit 1004 and secures the surgical bandage 1000 about the medical device (not shown). The tape strip 1040/flap 1014 may be of any dimensions necessary to close the slit 1004.

FIGS. 10i-10j are top views of the medical device securing mechanisms of the surgical bandage. The disposable surgical bandage 1000 includes an outer surface 1002 having a slit 1004 extending from an outer peripheral edge 1006 to an inner peripheral edge 1008 that forms an aperture 1010 to accommodate a medical device 1018.

FIG. 10i illustrates a device securing mechanism. A tape strip 1040/flap 1014 is positioned about the right portion 1028 of the slit 1004 and contacts the left portion 1026 of the slit 1004. The tape strip 1040/flap 1014 closes the slit 1004 and secures the surgical bandage 1000 about the medical device (not shown). The tape strip 1040/flap 1014 may be of any dimensions necessary to close the slit 1004. A first device securing strap 1042 is positioned on the outer surface 1002 to loop around the medical device 1018 and attach to the outer surface 1002. Similarly, a second device securing strap 1044 is also positioned on the outer surface 1002 to loop around the medical device 1018 and attach to the outer surface 1002. The first device securing strap 1042, the second device securing strap 1044 or both may be constructed from any applicable materials and be of any width, length and thickness.

FIG. 10j illustrates a device securing mechanism. A tape strip 1040/flap 1014 is positioned about the right portion 1028 of the slit 1004 and contacts the left portion 1026 of the slit 1004. The tape strip 1040/flap 1014 closes the slit 1004 and secures the surgical bandage 1000 about the medical device (not shown). The tape strip 1040/flap 1014 may be of any dimensions necessary to close the slit 1004. A device securing flap 1046 is positioned on the outer surface 1002 to fold around the medical device 1018 and itself to secure the medical device 1018.

FIG. 10k is a side view of the disposable surgical bandage 1000 to accommodate a medical device 1018 extending from an incision in a patient. The disposable surgical bandage 1000 includes a device securing flap 1046 positioned to cover the medical device 1018 and attach to the outer surface 1002. The device securing flap 1046 may be placed around the medical device 1018 and affixed to itself to form a conical shape. The device securing flap 1046 may then be attached to the outer surface 1002.

The present invention may use a variety of closure and affixing mechanisms (e.g., a hook and loop fastener system, fasteners, catches, snaps, loops, ties, clamps, connectors, couplers, links, bands, releasable adhesive, tape, glue, epoxy, adhesives (e.g., cyanoacrylates), tissue sealants and/or any other releasable mechanism for attachment, although the most commonly used is tape or adhesive to secured the material. A two-sided tape may be applied to one surface leaving the other side of the tape protected until ready for use. The adhesive side may have a separate protective layer that may be peeled away so that the adhesive can be applied to an appropriate surface. The protective layer prevents the adhesive from accidentally sticking to an unintended surface. The protective layer will also serve to help maintain the adhesiveness of the adhesive. This protective layer can be used for any closure or affixing mechanism, tape, glue, epoxy, and so forth.

In addition, the present invention may be used in conjunction with a transdermal device, a reservoir and/or impregnation adapted to retain during storage and release in operation one or more bioactive agents, e.g., analgesic, anti-allergenics, antipyretics, acetonide anti-inflammatory agents, antimicrobial agents, antibacterial agents, antifungal agents, antimycotic agents, antiviral agents, mixtures and combinations thereof. The present invention may include a coating layer (e.g., polymeric) on part or all of the surfaces that contains one or more bioactive agents, such as antibiotics.

Analgesic anti-inflammatory agents include for example, acetaminophen, aspirin, salicylic acid, methyl salicylate, choline salicylate, glycol salicylate, 1-menthol, camphor, mefenamic acid, fluphenamic acid, indomethacin, diclofenac, alclofenac, ibuprofen, ketoprofen, naproxene, pranoprofen, fenoprofen, sulindac, fenbufen, clidanac, flurbiprofen, indoprofen, protizidic acid, fentiazac, tolmetin, tiaprofenic acid, bendazac, bufexamac, piroxicam, phenylbutazone, oxyphenbutazone, clofezone, pentazocine, mepirizole, and the like.

Anti-allergenics include for example, antazoline, methapyrilene, chlorpheniramine, pyrilamine, pheniramine, and the like. Antipyretics include for example, aspirin, salicylamide, non-steroidal anti-inflammatory agents, and the like.

Acetonide anti-inflammatory agents include for example, hydrocortisone, cortisone, dexamethasone, fluocinolone, triamcinolone, medrysone, prednisolone, flurandrenolide, prednisone, halcinonide, methylprednisolone, fludrocortisone, corticosterone, paramethasone, betamethasone, ibuprophen, naproxen, fenoprofen, fenbufen, flurbiprofen, indoprofen, ketoprofen, suprofen, indomethacin, piroxicam, aspirin, salicylic acid, diflunisal, methyl salicylate, phenylbutazone, sulindac, mefenamic acid, meclofenamate sodium, tolmetin, and the like.

Antimicrobial agents include for example, antibacterial agents, antifungal agents, antimycotic agents and antiviral agents; tetracyclines such as, oxytetracycline, penicillins, such as, ampicillin, cephalosporins such as, cefalotin, aminoglycosides, such as, kanamycin, macrolides such as, erythromycin, chloramphenicol, iodides, nitrofrantoin, nystatin, amphotericin, fradiomycin, sulfonamides, purrolnitrin, clotrimazole, miconazole chloramphenicol, sulfacetamide, sulfamethazine, sulfadiazine, sulfamerazine, sulfamethizole and sulfisoxazole; antivirals, including idoxuridine; clarithromycin; and other anti-infectives including nitrofurazone; silver compound; a chlorhexidine gluconate compound and the like.

It is contemplated that any embodiment discussed in this specification can be implemented with respect to any method, kit, device or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the devices and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A disposable surgical bandage to accommodate a medical device extending from an incision in a patient comprising:
one or more absorbent layers each having a slit extending from an outer peripheral edge to an inner peripheral edge, wherein the inner peripheral edge forms an aperture to accommodate a medical device; and
one or more closure mechanisms to substantially secure the slit, whereby the medical device is positioned through the slit to the aperture and the bandage secured about the medical device for absorption of a wound exudates.

2. The device of claim 1, further comprising one or more outer surfaces disposed substantially coplanar to the one or more absorbent layers, wherein the one or more outer surfaces comprising an outer slit and an outer aperture that align with the slit and aperture to allow the medical device to be positioned through the slit to the aperture.

3. The device of claim 1, wherein the one or more closure mechanisms comprise a loop and hook fastener, an adhesive, a flap, a tape, a second surgical bandage or a combination thereof.

4. The device of claim 1, wherein the one or more closure mechanisms comprise a second absorbent layer having an opening extending orthogonally to the slit and extending from an outer peripheral edge to an inner peripheral edge, wherein orthogonal orientation of the opening to the slit secures the medical device and absorbs wound exudates.

5. The device of claim 1, further comprising an attachment mechanism disposed on the outer surface to adhere the bandage to the patient.

6. The device of claim 1, further comprising one or more foldable flaps attached to the outer surface to span securably the slit, one or more foldable flaps attached to the outer surface and attachable to the medical device.

7. The device of claim 1, wherein the aperture is rectangular, circular, polygonal, free-formed, triangular, oval or a combination thereof.

8. The device of claim 1, further comprising a gauze pledget spanning the aperture having a slot for receiving the medical device.

9. The device of claim 1, wherein said slit is linear, curved, zigzagged, angular, free-formed or a combination thereof.

10. The device of claim 1, further comprising removable protective shields on said adhesive.

11. The device of claim 1, wherein the one or more absorbent layers comprises a sterile gauze.

12. The device of claim 1, wherein the outer surface is designed to accommodate a percutaneous medical device.

13. A method of bandaging a medical device extending from an incision in a patient comprising the steps of:
moving a disposable surgical bandage over the incision in the patient to contact the medical device, wherein the disposable surgical bandage comprises an outer surface having a slit extending from an outer peripheral edge to an inner peripheral edge that forms an aperture to accommodate the medical device, one or more absorbent layers in contact with the outer surface and at least partially spans the aperture to accommodate the medical device and one or more closure mechanisms to substantially seal the slit;
sealing the one or more closure mechanisms to substantially seal the slit, thereby securing the surgical bandage around the medical device for absorption.

14. The method of claim 13, further comprising the step of adhering a flap over the slit to secure the surgical bandage around the medical device for absorption.

15. The method of claim 13, further comprising the step of adhering one or more flaps about the aperture to the medical device to secure the surgical bandage to the medical device for absorption.

16. The method of claim 13, wherein the one or more closure mechanisms comprise a loop and hook fastener, an adhesive, a flap, a tape, a second surgical bandage or a combination thereof.

17. The method of claim 13, further comprising removable protective shields to cover the closure mechanisms prior to use.

18. The method of claim 13, further comprising the step of attaching the outer surface of the surgical bandage to the patient.

19. The method of claim 13, wherein the aperture is rectangular, circular, polygonal, free-formed, triangular, oval or a combination thereof.

20. The method of claim 13, further comprising a gauze pledget spanning the aperture having a slot defined to receive the medical device.

21. The method of claim 13, wherein the medical device is a drainage tube, a trocart, a catheter, a chest drainage tube, or a percutaneous medical device.

22. The method of claim 13, wherein the one or more absorbent layers comprises a two or more of absorbent layers.

23. The method of claim 13, wherein the one or more absorbent layers comprises a sterile gauze.

24. A disposable surgical bandage to accommodate a medical device extending from an incision in a patient comprising:
an outer surface comprising a slit extending from an outer peripheral edge to an inner peripheral edge, wherein the inner peripheral edge forms an aperture to accommodate a medical device;
one or more absorbent layers disposed substantially coplanar with the outer surface and at least partially spans the aperture to accommodate the medical device; and
one or more closure mechanisms to substantially secure the slit, whereby the medical device is positioned through the slit to the aperture and the bandage secured about the medical device for absorption.
